(19) United States
(12) Patent Application Publication
(10) Pub. No.: US 2003/0203000 A1
(43) Pub. Date: Oct. 30, 2003

(54) Modulation of therapeutic agent release from a polymeric carrier using solvent-based techniques

(76) Inventors: Marlene C. Schwarz, Auburndale, MA (US); Douglas C. Shepard, Mansfield, MA (US)

Correspondence Address: MAYER, FORTKORT & WILLIAMS, PC, 251 NORTH AVENUE WEST, 2ND FLOOR, WESTFIELD, NJ, 07090, US

(21) Appl. No.: 10/131,745

(22) Filed: Apr. 24, 2002

(51) Int. Cl. A61K 9/14; A61M 31/00

(52) U.S. Cl. 424/423; 424/486; 604/500

(57) ABSTRACT

A method of modulating a rate of release of a therapeutic agent from a medical device is provided. The method comprises: (a) providing a solution comprising a therapeutic agent, a polymer and a solvent system; and (b) forming a therapeutic-agent-loaded polymeric carrier for the medical device by evaporating the solvent system, such that the rate of release is modulated by changing the composition of the solvent system. The composition of the solvent system can be changed in a number ways, including adding solvent species to the solvent system, removing solvent species from the solvent system, both adding and removing solvent species from the solvent system. The solvent system can also be changed by varying the ratio of solvent species within the solvent system.

### FIELD OF THE INVENTION

The present invention relates to methods for controlling delivery of a therapeutic agent from a polymeric carrier.

### BACKGROUND OF THE INVENTION

Numerous medical devices have been developed for the localized delivery of therapeutic agents to bodily tissue.

In accordance with one delivery strategy, a therapeutic agent is provided within a polymeric carrier that is associated with a medical device. Once the medical device is placed at the desired location upon or within the body, the therapeutic agent diffuses from the polymeric carrier. In this way, delivery of the therapeutic agent to bodily tissue is achieved.

The desired release profile for the therapeutic agent is dependent upon the particular treatment at hand, including the specific condition being treated/prevented, the specific therapeutic agent selected, the specific site of administration, and so forth.

It is therefore beneficial to have the means to adjust the release profile of therapeutic agent.

### SUMMARY OF THE INVENTION

The above and other needs of the prior art are met by the present invention, which is directed to a novel solvent-based strategy whereby the release profile of a therapeutic agent from a therapeutic-agent-loaded polymeric carrier is modulated.

According to an embodiment of the invention, a method of modulating a rate of release of a therapeutic agent from a medical device is provided. The method comprises: (a) providing a solution comprising a therapeutic agent, a polymer and a solvent system; and (b) forming a therapeutic-agent-loaded polymeric carrier for the medical device by evaporating the solvent system. The rate of release is modulated by changing the composition of the solvent system.

The composition of the solvent system can be changed in a number ways, including adding solvent species to the solvent system, removing solvent species from the solvent system, both adding and removing solvent species from the solvent system. The solvent system can also be changed by varying the ratio of solvent species within the solvent system.

Medical devices that can be made by this method include implantable or insertable medical devices, for example, implantable vascular medical devices. Preferably, the polymeric carrier is incorporated into the medical device as a coating over at least a portion of the medical device.

In some preferred embodiments, the polymeric carrier includes a polymer blend. In other preferred embodiments, the polymeric carrier includes a block copolymer. Preferred block copolymers are those comprising at least one polyolefin block and at least one polymethacrylate block or polyaromatic block. More preferably, the block copolymers comprise at least one block of polyisobutylene and at least one block of polystyrene or a polystyrene derivative.

As a specific example, a method is provided, which comprises: (a) providing a solution that further comprises (i) a block copolymer having at least one block of polyisobutylene and at least one block of polystyrene or a polystyrene derivative, (ii) paclitaxel and (iii) a solvent system comprising toluene and tetrahydrofuran; and (b) forming a therapeutic-agent-loaded polymeric carrier for the medical device by evaporating the solvent system. In this example, the release rate of the paclitaxel from the polymer carrier after solvent system evaporation is modulated in a predictable fashion by varying the amount of toluene relative to tetrahydrofuran within the solvent system. For example, the release rate of the paclitaxel from the polymer carrier is decreased by increasing the amount of toluene relative to tetrahydrofuran within the solvent system.

An advantage of the present invention is that it provides an effective method for controlling the release profile of a therapeutic agent from a therapeutic-agent-loaded polymeric carrier.

These and other embodiments and advantages of the present invention will become immediately apparent to those of ordinary skill in the art upon review of the Detailed Description and Claims to follow.

### BRIEF DESCRIPTION OF THE DRAWINGS

FIG. 1 is a plot of cumulative paclitaxel release as a function of time for various solvent systems consisting of tetrahydrofuran and toluene.

### DETAILED DESCRIPTION OF THE INVENTION

According to an embodiment of the present invention, release of a therapeutic agent from a therapeutic-agent-loaded polymeric carrier is modulated by varying the characteristics of the solvent system that is used to formulate the carrier.

A therapeutic-agent-loaded polymeric carrier formed in accordance with the present invention is preferably associated with a medical device to effect delivery of the therapeutic agent. For example, the therapeutic-agent-loaded polymeric carrier can constitute the entirety of the medical device or just a portion of the medical device. Portions of medical devices for which the therapeutic-agent-loaded polymeric carriers of the present invention find use include any fraction of a medical device, such as medical device coatings, medical device components, portions of medical device components and so forth.

In many preferred embodiments, a therapeutic-agent-loaded polymeric carrier is provided in the form of a coating on a medical device surface, including internal and/or external surfaces. The medical device surface or surfaces upon which the therapeutic-agent-loaded polymeric carrier is disposed can be formed from a wide variety of materials, including glasses, metals, polymers, ceramics and combinations thereof.

Preferred medical devices for use in conjunction with the present invention include catheters (preferably vascular catheters such as balloon catheters), guide wires, balloons, filters (e.g., vena cava filters), vascular stents, non-vascular stents (e.g., esophageal stents), stent grafts, cerebral stents, cerebral aneurysm filler coils (including GDC—Guglilmi detachable coils—and metal coils), vascular grafts, myocardial plugs, pacemaker leads and heart valves. The therapeutic-agent-loaded polymeric carriers of the present invention can also be used in connection with intraluminal paving systems and in connection with composites for aneurysm fillers.

The medical devices contemplated for use in connection with the present invention include drug delivery medical devices that are used for either systemic treatment or for the treatment of any mammalian tissue or organ. Non-limiting examples are tumors; organs including but not limited to the heart, lung, brain, liver, kidney, bladder, urethra and ureters, eye, intestines, stomach, pancreas, ovary, and prostate; skeletal muscle; smooth muscle; breast; cartilage; and bone.

Medical devices comprising therapeutic-agent-loaded polymeric carriers made in accordance with the present invention can be placed in a wide variety of bodily locations for contact with bodily tissue and/or fluid. Some preferred placement locations include the coronary vasculature or peripheral vascular system (referred to collectively herein as “the vasculature”), esophagus, trachea, colon, biliary tract, urinary tract, prostate and brain.

In some instances, it may be desirable to temporarily enclose the therapeutic-agent-loaded polymeric carrier to prevent initiation of release before the medical device reaches its ultimate placement site. As a specific example, a coated stent or catheter comprising a therapeutic-agent-loaded polymeric carrier can be covered with a sheath during insertion into the body to prevent premature therapeutic agent release.

Therapeutic agents useful in connection with the present invention include essentially any therapeutic agent that is compatible with solvent-based techniques and with the selected polymeric carrier (e.g., is not adversely affected by the polymeric carrier and can be released from the polymeric carrier). Therapeutic agents may be used singly or in combination.

“Therapeutic agents”, “pharmaceutically active agents”, “pharmaceutically active materials”, “drugs” and other related terms may be used interchangeably herein and include genetic therapeutic agents, non-genetic therapeutic agents and cells.

Exemplary non-genetic therapeutic agents include: (a) anti-thrombotic agents such as heparin, heparin derivatives, urokinase, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; and (o) agents that interfere with endogenous vascoactive mechanisms.

Exemplary genetic therapeutic agents include anti-sense DNA and RNA as well as DNA coding for: (a) anti-sense RNA, (b) tRNA or rRNA to replace defective or deficient endogenous molecules, (c) angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin-like growth factor, (d) cell cycle inhibitors including CD inhibitors, and (e) thymidine kinase (“TK”) and other agents useful for interfering with cell proliferation. Also of interest is DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors of interest for delivery of genetic therapeutic agents include (a) plasmids, (b) viral vectors such as adenovirus, adenoassociated virus and lentivirus, and (c) non-viral vectors such as lipids, liposomes and cationic lipids.

Cells include cells of human origin (autologous or allogeneic), including stem cells, or from an animal source (xenogeneic), which can be genetically engineered if desired to deliver proteins of interest.

A number of the above therapeutic agents and several others have also been identified as candidates for vascular treatment regimens, for example, as agents targeting restenosis. Such agents are appropriate for the practice of the present invention and include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) ACE inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, fluvastatin, simvastatin and cerivastatin, (u) fish oils and omega-3-fatty acids, (v) free-radical scavengers/antioxidants such as probucol, vitamins C and E, ebselen, trans-retinoic acid and SOD mimics, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) MMP pathway inhibitors such as marimastat, ilomastat and metastat, (y) cell motility inhibitors such as cytochalasin B, (z) antiproliferative/antineoplastic agents including antimetabolites such as purine analogs(6-mercaptopurine), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, paclitaxel and epothilone), caspase activators, proteasome inhibitors, angiogenesis inhibitors (e.g., endostatin, angiostatin and squalamine), rapamycin, cerivastatin, flavopiridol and suranin, (aa) matrix deposition/organization pathway inhibitors such as halofuginone or other quinazolinone derivatives and tranilast, (bb) endothelialization facilitators such as VEGF and RGD peptide, and (cc) blood rheology modulators such as pentoxifylline.

Several of the above and numerous additional therapeutic agents appropriate for the practice of the present invention are also disclosed in U.S. Pat. No. 5,733,925 assigned to NeoRx Corporation, the entire disclosure of which is incorporated by reference.

A wide range of therapeutic agent loadings can be used in connection with the above polymeric carriers, with the amount of loading being readily determined by those of ordinary skill in the art and ultimately depending upon the condition to be treated, the nature of the therapeutic agent itself, the means by which the therapeutic-agent-loaded polymeric carrier is administered to the intended subject, and so forth. The loaded polymeric carrier will frequently comprise from 1% or less to 70 wt % or more therapeutic agent.

Polymers for use in forming the polymeric carrier include essentially any polymer (including polymer blends) that allows for the release of the therapeutic agent, can be dissolved in a solvent system, and is compatible with solvent-based techniques, with the therapeutic agent, with the medical device, with its site of administration, and so forth. The polymers may be crosslinked or uncrosslinked, linear or branched, natural or synthetic, thermoplastic or thermosetting.

Exemplary polymers include the following: polycarboxylic acid polymers and copolymers including polyacrylic acids (e.g., acrylic latex dispersions and various polyacrylic acid products such as HYDROPLUS, available from Boston Scientific Corporation, Natick Mass. and described in U.S. Pat. No. 5,091,205, the disclosure of which is hereby incorporated herein by reference, and HYDROPASS, also available from Boston Scientific Corporation); acetal polymers and copolymers; acrylate and methacrylate polymers and copolymers; cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydoxyalkyl celluloses; polyoxymethylene polymers and copolymers; polyimide polymers and copolymers such as polyether block imides, polybismaleinimides, polbyamidimides, polyesterimides, and polyetherimides; polysulfone polymers and copolymers including polyarylsulfones and polyethersulfones; polyamide polymers and copolymers including nylon 6,6, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); anhydride polymers and copolymers including maleic anhydride polymers; polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, polystyrenes, styrene-butadiene copolymers, acrylonitrile-styrene copolymers, acrylonitrile-butadiene-styrene copolymers, styrene-butadiene-styrene copolymers and styrene-isobutylene-styrene copolymers, polyvinyl ketones, polyvinylcarbazoles, and polyvinyl esters such as polyvinyl acetates; polybenzimidazoles; ionomers; polyalkyl oxide polymers and copolymers including polyethylene oxides (PEO); glycosaminoglycans; polyesters including polyethylene terephthalates and aliphatic polyesters such as polymers and copolymers of lactide (which includes lactic acid as well as d-,1- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyisocyanates (e.g., U.S. Pat. No. 5,091,205 describes medical devices coated with one or more polyisocyanates such that the devices become instantly lubricious when exposed to body fluids); polyolefin polymers and copolymers, including polyalkylenes such as polypropylenes, polyethylenes (low and high density, low and high molecular weight), polybutylenes (such as polybut-1-ene and polyisobutylene), poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; polyurethanes (e.g., BAYHYDROL polyurethane dispersions); p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as blends and copolymers of the above.

Preferred polymers for use in connection with the present invention are block copolymers having at least two polymeric blocks A and B. Examples of such block copolymers include the following: (a) BA (linear diblock), (b) BAB or ABA (linear triblock), (c) B(AB)ₙ or A(BA)ₙ (linear alternating block), or (d) X-(AB)ₙ or X-(BA)ₙ (includes diblock, triblock and other radial block copolymers), where n is a positive whole number and X is a starting seed molecule.

One specific preferred group of polymers have X-(AB)ₙ structures, which are frequently referred to as diblock copolymers and triblock copolymers where n=1 and n=2, respectively (this terminology disregards the presence of the starting seed molecule, for example, treating A-X-A as a single A block with the triblock therefore denoted as BAB). Where n=3 or more, these structures are commonly referred to as star-shaped block copolymers.

The A blocks are preferably soft elastomeric components which are based upon one or more polyolefins, more preferably a polyolefinic block having alternating quaternary and secondary carbons of the general formulation: —(CRR′—CH₂)ₙ—, where R and R′ are linear or branched aliphatic groups such as methyl, ethyl, propyl, isopropyl, butyl, isobutyl and so forth, or cyclic aliphatic groups such as cyclohexane, cyclopentane, and the like, with and without pendant groups. Polymers of isobutylene,

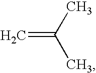

(i.e., polymers where R and R′ are the same and are methyl groups) are more preferred.

The B blocks are preferably hard thermoplastic blocks that, when combined with the soft A blocks, are capable of, inter alia, altering or adjusting the hardness of the resulting copolymer to achieve a desired combination of qualities. Preferred B blocks are polymers of methacrylates or polymers of vinyl aromatics. More preferred B blocks are (a) made from monomers of styrene

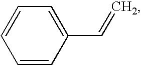

styrene derivatives (e.g., α-methylstyrene, ring-alkylated styrenes or ring-halogenated styrenes) or mixtures of the same or are (b) made from monomers of methylmethacrylate, ethylmethacrylate hydroxyethyl methacrylate or mixtures of the same.

Particularly preferred polymers for use in connection with the present invention include copolymers of polyisobutylene with polystyrene or polymethylstyrene, more preferably polystyrene-polyisobutylene-polystyrene triblock copolymers. These polymers are described, for example, in U.S. Pat. No. 5,741,331, U.S. Pat. No. 4,946,899 and U.S. Ser. No. 09/734,639, each of which is hereby incorporated by reference in its entirety.

The polymers can also be used in connection with further auxiliary materials to achieve a desired result. Such auxiliary materials include binders, blending agents, and so forth.

In some cases, it may be useful to coat the therapeutic-agent-loaded polymeric carrier with an additional polymer layer, which may serve, for example, as a boundary layer to further retard diffusion of the therapeutic agent. The material constituting additional polymer layer may or may not be of the same material as the polymeric carrier and can be selected from those polymers listed above.

In general, the therapeutic-agent-loaded polymeric carriers of the present invention are formed using any of a number of known solvent-based techniques in which the polymer and therapeutic agent are first dissolved in a solvent, after which the resulting solution is used to form the loaded polymeric carrier. Hence, in the present invention, the therapeutic agent is loaded concurrently with polymeric carrier formation.

Preferred solvent-based techniques of this nature include, but are not limited to, solvent casting, spin coating, web coating, solvent spraying, dipping, coating via air suspension and mechanical suspension techniques, ink jet techniques, electrostatic techniques, and combinations of these processes.

In some of these techniques, a solution containing solvent, therapeutic agent and polymer is applied to a substrate to form therapeutic-agent-loaded polymeric carrier. The substrate can be, for example, all or a portion of a medical device to which a therapeutic-agent-loaded polymeric carrier is applied as a coating. The substrate can also be, for example, a template from which the therapeutic-agent-loaded polymeric carrier is removed after solvent elimination. Such template-based techniques are particularly appropriate for forming simple objects such as sheets, tubes, cylinders and so forth, which can be easily removed from a template substrate.

In other techniques, for example, fiber forming, the therapeutic-agent-loaded polymeric carrier is formed without the aid of a substrate.

Where appropriate, techniques such as those listed above can be repeated or combined to build up a therapeutic-agent-loaded polymeric carrier to a desired thickness. Polymeric carrier thickness can be varied in other ways as well. For example, in one preferred process, solvent spraying, coating thickness can be increased by modification of coating process parameters, including increasing flow rate, slowing the movement between the device or template to be coated and the spray nozzle, providing repeated passes and so forth.

After the therapeutic-agent-loaded polymeric carrier is formed (using one of the above processes, for example), it is preferably dried to remove the solvents. In the case of a coating, the coating typically conforms to the underlying surface during the drying process, with the therapeutic agent being incorporated into the polymeric coated layer.

In the method of the present invention, release of the therapeutic agent from the polymeric carrier is modulated by varying the characteristics of the solvent system that is used to form the therapeutic-agent-loaded polymeric carrier.

Ideally, the release characteristics of interest are the release characteristics within the subject, for example, a mammalian subject. However, it is well known in the art to test the release characteristics within an experimental system that gives a good indication of the actual release characteristics within the subject. For example, aqueous buffer systems are commonly used for testing release of therapeutic agents from vascular devices.

In general the solvent system that is selected contains one or more solvent species. The solvent system is a good solvent for the polymer and for the therapeutic agent.

In addition to their ability to contribute to the solubility of the polymeric and therapeutic constituents (and their compatibility with the these constituents), the particular solvent species that make up the solvent system may also be selected based on other characteristics including drying rate and surface tension.

Solvent species that can be used in connection with the present invention include any combination of one or more of the following: (a) water, (b) alkanes such as ethane, hexane, octane, cyclohexane, heptane, isohexane, butane, pentane, isopentane, 2,2,4-trimethlypentane, nonane, decane, dodecane, hexadecane, eicosane, methylcyclohexane, cis-decahydronaphthalene and trans-decahydronaphthalene, (c) aromatic species such as benzene, toluene, xylene(s), naphthalene, styrene, ethylbenzene, 1-methylnaphthalene, 1,3,5-trimethylbenzene, tetrahydronaphthalene, diphenyl and 1,4-diethylbenzene, (d) halohydrocarbons including (i) chlorohyhdrocarbons such as chloroform, methyl chloride, dichloromethane, 1,1-dichloroethylene, ethylene dichloride, ethylidene chloride, propyl chloride, cyclohexyl chloride, 1,1,1-trichloroethane, perchloroethylene, trichloroethylene, butyl chloride, carbon tetrachloride, tetrachloroethylene, chlorobenzene, o-dichlorobenzene, benzyl chloride, trichlorobiphenyl, methylcyclohexane, 1,1,2,2-tetrachloroethane (ii) fluorinated halogenated species such as chlorodiflouoromethane, dichlorofluoromethane, dichlorodifluoromethane, trichlorofluoromethane, 1,2-dichlorotetrafluoroethane, 1,1,2-trichlorotrifluoroethane, perfluor(methylcyclohexane), perfluor(dimethylcyclohexane) and (iii) other halohydrocarbons such as ethyl bromide, ethylidene bromide, ethylene dibromide, tribromomethane, bromotrifluoromethane, 1,1,2,2-tetrabromoethane, bromobenzene, bromochloromethane, 1-bromonaphthalene, methyl iodide, methylene diiodide (e) acid aldehydes/anhydrides such as acetaldehyde, furfural, butyraldehyde, benzaldehyde, acetyl chloride, succinic anhydride and acetic anhydride, (f) alcohols including (i) phenols such as phenol, 1,3-benzenediol, m-cresol, o-methoxyphenol, methyl salicylate and nonylphenol, (ii) polyhydric alcohols such as ethylene glycol, glycerol, propylene glycol, 1,3-butanediol, diethylene glycol, triethylene glycol, hexylene glycol and dipropylene glycol, and (iii) other alcohols such as methanol, ethanol, ethylene cyanohydrin, allyl alcohol, 1-propanol, 2-propanol, 3-chloropropanol, furfuryl alcohol, 1-butanol, 2-butanol, benzyl alcohol, isobutanol, cyclohexanol, 1-pentanol, 2-ethyl-1-butanol, diacetone alcohol, 1,3-dimethyl-1-butanol, ethyl lactate, butyl lactate, ethylene glycol monomethyl ether, ethylene glycol monoethyl ether, diethylene glycol monomethyl ether, diethylene glycol monoethyl ether, ethylene glycol monobutyl ether, 2-ethyl-1-hexanol, 1-octanol, 2-octanol, diethylene glycol monobutyl ether, 1-decanol, 1-tridecyl alcohol, nonyl-phenoxy ethanol, oleyl alcohol, triethylene glycol mono-oleyl ether, (g) ethers such as, epichlorohydrin, furan, 1,4-dioxane, dimethoxymethane, diethyl ether, bis-(2-chloroethyl)ether, anisole, di-(2-methoxyethyl)ether, dibenzyl ether, di-(2-chloroisopropyl)ether, bis-(m-phenoxyphenol)ether, dimethyl ether and tetrahydrofuran, (h) ketones, such as acetone, cylohexanone, isophorone, diethyl ketone, mesityl oxide, acetophenone, methyl ethyl ketone, methyl isoamyl ketone, methyl isobutyl ketone, and methyl propyl ketone, (i) acids such as formic acid, acetic acid, benzoic acid, butyric acid, octanoic acid, oleic acid, stearic acid, (j) esters/acetates such as ethylene carbonate, butyrolactone, propylene-1,2-carbonate, ethyl chloroformate, ethyl acetate, trimethyl phosphate, diethyl carbonate, diethyl sulfate, ethyl formate, methyl acetate, n-butyl acetate, isobutyl acetate, t-butyl acetate, 2-ethoxyethyl acetate, isoamyl acetate, dimethyl phthalate, ethyl cinnamate, triethyl phosphate, diethyl phosphate, butyl benzyl phthalate, dibutyl phthalate, diethyl phthalate, tricrysyl phosphate, tributyl phosphate, dibutyl sebacate, methyl oleate, dioctyl phthalate, dibutyl stearate isopropyl acetate, isobutyl isobutyrate, n-propyl acetate and n-butyl propionate, (k) nitrogen compounds such as acetonitrile, acrylonitrile, propionitrile, butyronitrile, nitromethane, nitroethane, 2-nitropropane, nitrobenzene, ethanolamine, ethylenediamine, 1,1-dimethylhydrazine, 2-pyrrolidone, pyridine, propylamine, morpholine, analine, n-methyl-2-pyrrolidone, butylamine, diethylamine, cyclohexylamine, quinoline, dipropylamine, formamide, n,n-dimethylformamide, n,n-dimethylacetamide, tetramethylurea, hexamethyl phosphoramide, diethylenetriamine, triethylamine and triethanolamine, and (1) sulfur compounds such as carbon disulfide, dimethylsulfoxide, ethanethiol, dimethyl sulfone and diethyl sulfide.

In addition to meeting the above criteria, the solvent system is also selected such that release of therapeutic agent from the polymeric carrier can be modulated by changing the makeup of the solvent system. For example, the makeup of the solvent system can be changed by adding one or more solvent species to the solvent system, by removing one or more solvent species from the solvent system, or both adding and removing solvent species from the solvent system. Moreover, even though the particular species making up the solvent system may remain unchanged, the ratio of the solvent species relative to one another can be changed.

In many preferred embodiments, the ratio of polymer to therapeutic agent is held constant as the solvent system is changed.

It is also noted that particular species of the solvent system may also be selected to impart characteristics to the therapeutic-agent-loaded polymeric carrier besides release characteristics, including biocompatibility, bioerosion and biodegradation.

A specific example of the present invention is presented in the Example below, in which the therapeutic agent is paclitaxel and the polymer is a polystyrene-polyisobutylene-polystyrene tri-block copolymer. As seen from this example, the release rate can be varied by adding solvent species, removing solvent species, and changing the ratio of solvent species. For instance, the data in the Example suggest that where a tetrahydrofuran (THF) solvent system is selected, the release rate can be reduced by adding toluene as a solvent species, and vice versa. Moreover, within a toluene/THF solvent system, the rate of release can be reduced by increasing the ratio of toluene relative to THF in the system. Conversely, the release rate can be increased by increasing the amount THF relative to toluene.

Although not wishing to be bound by theory, it is generally believed that effectiveness of the method of the invention is due, at least in part, to changes in the distribution of the polymer and therapeutic agent within the resulting product. For example, depending upon the solvent system selected, as the solvent evaporates, (a) the therapeutic agent may be dissolved within the polymer phase, (b) the therapeutic agent may form a phase of its own that is distinct from the polymer phase, or (c) a portion of the therapeutic agent may be dissolved within the polymer phase and a portion may form its own phase. Moreover, the therapeutic agent may preferentially occupy a given region of the polymer carrier. For example, therapeutic agent may preferentially occupy the surface, a region just below the surface, or the bulk of the polymeric carrier. This, in turn, influences the release characteristics of the therapeutic agent from the loaded polymeric carrier.

In addition, the situation is more complex where the therapeutic-agent-loaded polymeric carrier is formed from a copolymer that contains polymer blocks of varying polarity, or where a polymer blend is selected which contains distinct polymer species of varying polarity. Under these circumstances, phase separation of the polymer blocks/polymer components from one another can occur as the solvent evaporates, resulting the formation of distinct polymer domains (phases), which in turn influence the distribution of the therapeutic agent and hence the release rate. The formation of distinct polymer domains may also result in the migration of one of the domains to the surface, affecting the surface properties of the polymer carrier that is formed, including the surface tension of the resulting layer and the biocompatibility of the same.

The invention is further described with reference to the following non-limiting Example.

### EXAMPLE

A solution is provided that contains the following: 99 wt % solvent system, 0.25 wt % paclitaxel and 0.75 wt % block copolymer. The copolymer is synthesized using known techniques such as those described in U.S. Pat. No. 5,741,331, U.S. Pat. No. 4,946,899 and U.S. Ser. No. 09/734,639. The solvent system consists of tetrahydrofuran (THF) and toluene, which are provided in varying ratios in this example. The solution is provided by (1) mixing the paclitaxel and tetrahydrofuran, (2) adding the copolymer, (3) adding the toluene, (4) thoroughly mixing (e.g., overnight), and (5) filtering.

The solution is then placed in a syringe pump and fed to a spray nozzle. A stent is mounted onto a holding device parallel to the nozzle and, if desired, rotated to ensure uniform coverage. Depending on the spray equipment used, either the component or spray nozzle can be moved while spraying such that the nozzle moves along the component while spraying for one or more passes.

After a coating is formed in this fashion, it is dried, for example, by placing it in a preheated oven for 30 minutes at 65° C., followed by 3 hours at 70° C.

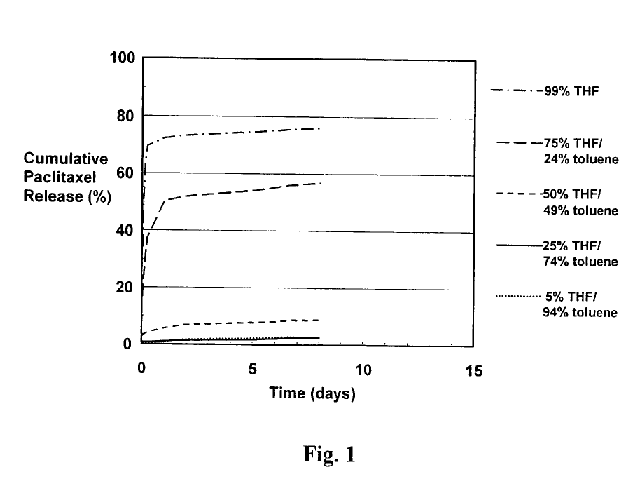

Three coated stents are formed in this manner using (in addition to 0.25 wt % paclitaxel and 0.75 wt % block copolymer) the following solvent species in the following relative amounts: (T) 99 wt % THF, (2) 75 wt % THF, 24 wt % toluene, (3) 50 wt % THF, 49 wt % toluene, (4) 25 wt % THF, 74 wt % toluene, (5) 5 wt % THF, 94 wt % toluene. Release rate as a function of time and cumulative release as a function of time (referred to herein as the “release profile”) were then measured in PBS with 0.5% Tweeng 20 (polyoxyethylene(20) sorbitan monolaurate) available from Sigma-Aldrich. The results are graphically illustrated in FIG. 1.

As can be seen from this figure, where THF is selected as the solvent species for the solvent system, paclitaxel release can be reduced by the addition of toluene to the system. Moreover, paclitaxel release varies with the ratio of THF to toluene within the solvent system. Higher THF-to-toluene ratios result in more accelerated paclitaxel release, while lower THF-to-toluene ratios result in more extended paclitaxel release.

As noted above, and without wishing to be bound by theory, it is believed that changes in the solvent system result in changes in the distribution of the paclitaxel within the polymeric carrier, which in turn alters the release characteristics of the paclitaxel from the polymeric carrier.

Although various embodiments are specifically illustrated and described herein, it will be appreciated that modifications and variations of the present invention are covered by the above teachings and are within the purview of the appended claims without departing from the spirit and intended scope of the invention.

In the claims:

1. A method of modulating a rate of release of a therapeutic agent from a medical device, said method comprising: (a) providing a solution comprising a therapeutic agent, a block copolymer, and a solvent system; and (b) forming a therapeutic-agent-loaded polymeric carrier for said medical device by evaporating said solvent system, wherein said rate of release is modulated by changing the composition of said solvent system.

2. The method of claim 1, wherein said polymeric carrier is incorporated into said medical device as a coating over at least a portion of said medical device.

3. The method of claim 1, wherein said medical device is an implantable or insertable medical device.

4. The method of claim 1, wherein said medical device is an implantable vascular medical device.

5. The method of claim 1, wherein said composition of said solvent system is changed by adding solvent species to the solvent system.

6. The method of claim 1, wherein said composition of said solvent system is changed by removing solvent species from the solvent system.

7. The method of claim 1, wherein said composition of said solvent system is changed by both adding solvent species to the solvent system and removing solvent species from the solvent system.

8. The method of claim 1, wherein said solvent system comprises first and second solvent species, and wherein said composition of said solvent system is changed by changing the amount of said first solvent species relative to said second solvent species.

9. The method of claim 1, wherein said block copolymer comprises (a) at least one polyolefin block and (b) at least one polymethacrylate block or polyaromatic block.

10. The method of claim 1, wherein said block copolymer comprises (a) at least one block of polyisobutylene and (b) at least one block of polystyrene or a polystyrene derivative.

11. The method of claim 10, wherein said solvent system comprises toluene and tetrahydrofuran.

12. The method of claim 11, wherein said therapeutic agent is paclitaxel.

13. The method of claim 12, wherein said composition of said solvent system is changed by changing the amount of toluene relative to tetrahydrofuran.

14. A medical device formed by the method of claim 1.

15. A medical device formed by the method of claim 4.

16. A medical device formed by the method of claim 9.

17. A method of modulating a rate of release of a therapeutic agent from a medical device, said method comprising: (a) providing a solution comprising a therapeutic agent, a polymer, and a solvent system; and (b) forming a therapeutic-agent-loaded polymeric carrier for said medical device by evaporating said solvent system, wherein said rate of release is modulated by changing the composition of said solvent system.

18. The method of claim 17, wherein said medical device is an implantable vascular medical device.

19. The method of claim 17, wherein said polymer is a polymer blend.

20. A medical device formed by the method of claim 17.
